(19) United States
(12) Patent Application Publication
(10) Pub. No.: US 2018/0289530 A1
(43) Pub. Date: Oct. 11, 2018

(54) HEATING KINESIOLOGY TAPE

(71) Applicant: Rocktape, Inc., Campbell, CA (US)

(72) Inventors: Gregory van den Dries, Los Gatos, CA (US); Vitaly Vasilyevich Gashpar, San Jose, CA (US); Bruce Arnold Young, Le Mars, IA (US)

(73) Assignee: Rocktape, Inc., Campbell, CA (US)

(21) Appl. No.: 15/766,269

(22) PCT Filed: Oct. 15, 2016
(86) PCT No.: PCT/IB2016/056190

§ 371 Date: Apr. 10, 2018

Related U.S. Application Data
(60) Provisional application No. 62/250,385, filed on Nov. 3, 2015.

(51) Int. Cl. A61F 7/00; A61F 13/02; A61F 7/02

(52) U.S. Cl. CPC A61F 7/007; A61F 13/02; A61F 2013/00195; A61F 2007/0071; A61F 2007/0226; A61F 7/02

(57) ABSTRACT

A heating tape system includes an adhesive tape and a power supply. The adhesive tape is made of an anisotropically stretchable fabric material with a pressure sensitive adhesive fixed on one side of the fabric material, and a heating element coupled to the fabric material. A first load contact and a second load contact, both electrically coupled to the heating element and accessible from the opposite side of the fabric material, are also included on the adhesive tape. The power supply includes a source of electrical energy with a first source contact electrically coupled to one terminal of the source of electrical energy and adapted to be electrically coupled to the first load contact, and a second source contact electrically coupled to a second terminal of the source of electrical energy and adapted to be electrically coupled to the second load contact.

### CROSS-REFERENCE TO RELATED APPLICATIONS

This application claims priority to U.S. Provisional Patent Application No. 62/250,385 filed on Nov. 3, 2015 and entitled HEATING KINESIOLOGY TAPE, which incorporated by reference herein for any and all purposes.

### BACKGROUND

### Technical Field

The present subject matter relates to therapeutic tape for human application, and more specifically, to adhesive tape that has anisotropic stretch properties and a heating element.

### Background Art

Kinesiology tape, which is often a cloth-based self-adhesive tape, is a tape with anisotropic stretch properties, so that it is able to stretch much more in one direction, such as the length, than in the other direction, such as width. Traditionally, kinesiology tape is used to treat muscles and/or connective tissue that has been stressed in some way, but where there is no open wound. Kinesiology tape is applied to the individual in a stretched condition to provide a therapeutic benefit to the individual from the recoil effect of the elasticity of the tape. Kinesiology tape can be applied in many different configurations, depending on the tissue group being targeted and the intended effect, but in at least some situations, the tape is applied, in a stretched position along the area of interest. Once the tape has been applied, it is often rubbed to activate a pressure-sensitive adhesive. While kinesiology tape is sometimes provided in pre-cut sections for specific taping patterns, it is often provided in a bulk form, such as a roll that allows for individual strips of tape to be cut to an appropriate length as needed.

Heat is used as therapy for a variety of conditions. Therapeutic effects of heat therapy, which is also sometimes called thermotherapy, include increasing the extensibility of collagen tissues, decreasing joint stiffness, reducing pain, relieving muscle spasms, reducing inflammation, reducing edema, and increasing blood flow. Increasing blood flow helps provide proteins, nutrients, and oxygen to the affected part of the body for better healing. Heat therapy utilizes a wide variety of temperatures and treatment duration, but in many cases, lower temperatures applied for longer durations have been found to be more effective than brief applications of higher temperatures.

### BRIEF DESCRIPTION OF THE DRAWINGS

The accompanying drawings, which are incorporated in and constitute part of the specification, illustrate various embodiments. Together with the general description, the drawings serve to explain various principles. In the drawings:

FIG. 1A shows the skin-facing side of an embodiment of a heating kinesiology tape in an unstretched position;

FIG. 1B shows the non-skin-facing side of an embodiment of heating kinesiology tape in an unstretched position;

FIG. 1C shows the non-skin-facing side of an embodiment of heating kinesiology tape in a stretched position;

FIG. 2 shows a roll of an embodiment of heating kinesiology tape.

FIG. 3 shows an embodiment of heating kinesiology tape system used to treat a muscle on a human leg;

FIG. 4 is a schematic view of an embodiment of heating kinesiology tape with a power source;

FIG. 5A shows a cross-sectional view of an embodiment of a heating tape system;

FIG. 5B shows a cross-sectional view of an alternative embodiment of a heating tape system;

FIG. 6 is a flowchart of an embodiment of a method of applying heating kinesiology tape to a body;

FIG. 7 is a flowchart of an embodiment of a method of manufacturing heating kinesiology tape;

FIG. 8 shows an alternative embodiment of a heating kinesiology tape system; and

FIG. 9 shows another alternative embodiment of a heating kinesiology tape system.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures and components have been described at a relatively high-level, without detail, in order to avoid unnecessarily obscuring aspects of the present concepts. A number of descriptive terms and phrases are used in describing the various embodiments of this disclosure. These descriptive terms and phrases are used to convey a generally agreed upon meaning to those skilled in the art unless a different definition is given in this specification. Some descriptive terms and phrases are presented in the following paragraphs for clarity.

Kinesiology tape, as the term is used herein, refers to a fabric tape with anisotropic stretch qualities, so that the kinesiology tape can be stretched in one direction at least twice as much as in the orthogonal direction without significantly changing the unstretched dimensions of the kinesiology tape or its stretch attributes. An adhesive is affixed to one side of the kinesiology tape. The amount of stretch can vary between different types of kinesiology tape, but any kinesiology tape can stretch to at least 125% of its unstretched length in its stretchable direction without a significant change in the unstretched length of the kinesiology tape after it returns to an unstretched state. The ROCKTAPE® brand of kinesiology tape can be stretched up to about 180% of its unstretched length. The direction of the kinesiology tape (or any anisotropically stretchable material) that can be stretched more than other directions may be referred to as the stretchable direction, even though the tape may allow some amount of stretch in the other directions. Kinesiology tape can be stretched in its non-stretchable direction, or the direction orthogonal to the stretchable direction, to a much lesser extent than its stretchable direction without impacting its unstretched dimensions, with a typical kinesiology tape stretchable only up to about 110% or less in the non-stretchable direction.

The term “stretch” and its derivatives, as used herein, refers to lengthening a linear dimension of a material in a particular direction by applying a tensile force on the material in that direction. In some cases, other dimensions of the material may change, such as shorten, as the material lengthens in the stretch direction. As the term is used herein, once the tensile force on the material is removed, the material will return to about its original dimensions with no substantial change in the dimension of the material in the direction that the material was stretched. If the material does not return to about its original dimensions once the tensile force is removed, the material is deemed to have been overstretched instead of stretched.

Reference now is made in detail to the examples illustrated in the accompanying drawings and discussed below.

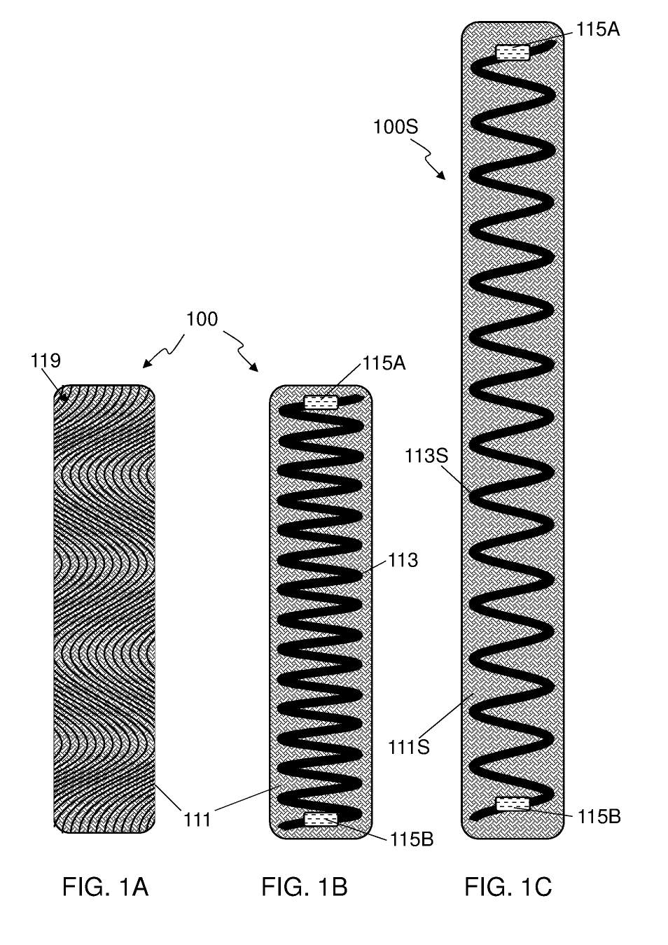

FIG. 1A shows the skin-facing side of an embodiment of a heating kinesiology tape 100 in an unstretched position. The heating kinesiology tape 100 can be an adhesive tape. The tape 100 includes a piece of anisotropically stretchable fabric material 111 having a first side shown in FIG. 1A and a second side, shown in FIG. 1B, opposite the first side. In at least some embodiments, the fabric 111 is breathable, allowing gasses and in some cases, fluids, to pass through the fabric 111. The fabric 111 may be made from any type of fiber, but in at least one embodiment, the fabric 111 comprises 97% cotton and 3% Nylon. The fabric 111 may be identical to, or similar to, fabric used in one of many well-known brands of kinesiology tape, such as the ROCKTAPE brand of kinesiology tapes.

In at least one embodiment, the long direction of the loom is the warp of the fabric 111 and forms the base part of the weave. The fabric 111 can have a warp of cotton that is coupled with 70 denier spandex, such as Lycra® brand spandex from Invista, or some other nylon-based material with similar stretch characteristics. By using these types of fabrics, the tape 100 has a warp direction which allows it to stretch along the longitudinal axis, thus the anisotropically stretchable fabric material is stretchable longitudinally. The fabric 111 can use a ring spun cotton in the waft (horizontal) direction. Using ring spun cotton versus typical cotton yarn can provide more heft to the ‘hand’ or feel of the tape making it feel more luxurious. By combining the spandex in the warp layer and ring spun cotton in the weft layer, the tape 100 can effectively stretch in one direction only and have similar stretch characteristics to skin. Some embodiments of the tape 100 can stretch from 110-180% of its original length. Some embodiments of the tape 100 can stretch up to 125% of its original length. Some embodiments of the tape 100 can stretch up to 140% of its original length. Some embodiments of the tape 100 can stretch up to 175% of its original length. Various embodiments can have a maximum stretch of any amount over 125%.

The adhesive tape 100 includes a pressure-sensitive adhesive 119 fixed to the first side of the fabric material 111. In embodiments, the adhesive 119 is applied to at least some of an exposed area of the first side of the fabric 111, the skin-facing side of the tape 100. While some embodiments may fully coat the exposed areas of the skin-facing side of the fabric 111 with the pressure-sensitive adhesive 119, other embodiments apply the pressure-sensitive adhesive 119 in pattern of alternating adhesive and non-adhesive areas such as the wave pattern shown. Other embodiments may use other patterns including, but not limited to, lines or rectangular areas, dots or circles, or any other type of pattern. The adhesive may be hypoallergenic and/or latex-free, such as an acrylic adhesive, and may be water resistant in some embodiments, to allow the heating kinesiology tape to remain adhered to a person's body even under moist or wet conditions, such as when the person is sweating heavily. In some embodiments, the adhesive 119 may be applied to the fabric at the rate of 65-72 grams per square meter. In at least one embodiment, the fabric 111 and pressure-sensitive adhesive 119 are identical to those of one of the types of ROCKTAPE brand kinesiology tape, such as the ROCKTAPE Active Recovery (AR) tapes or the ROCKTAPE H2O tapes. In some embodiments the adhesive 119 may be covered with a release paper to protect the adhesive 119 until the adhesive tape 100 is to be applied.

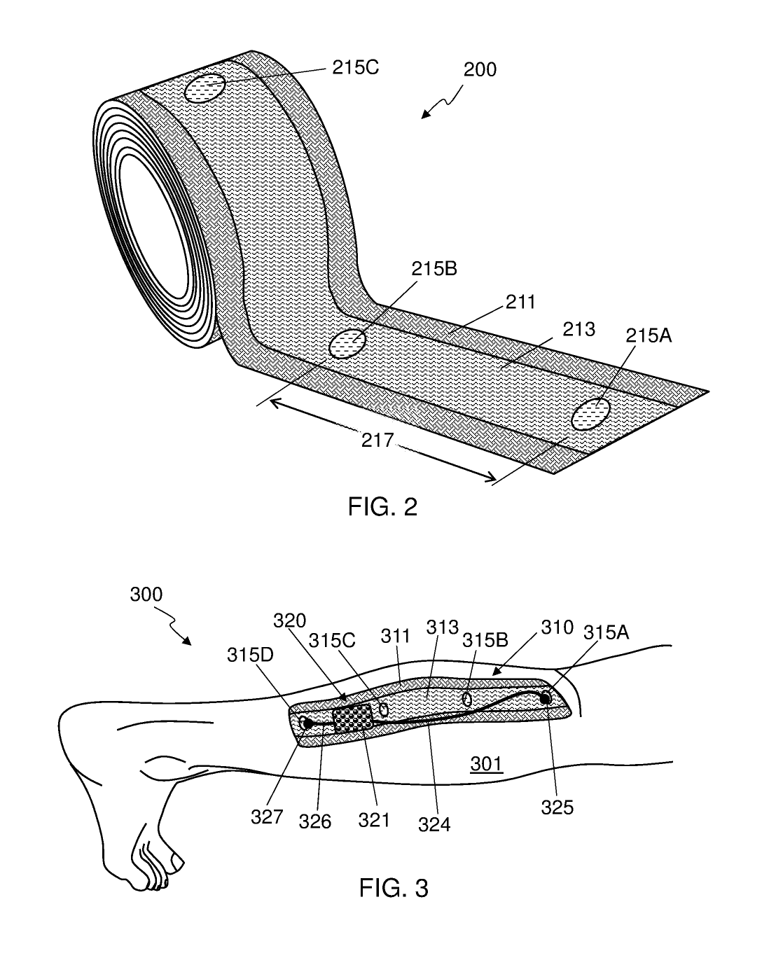

FIG. 1B shows the non-skin-facing side of an embodiment of a heating kinesiology tape 100 in an unstretched position. FIG. 1B shows the opposite side of the tape 100 of FIG. 1A. The tape 100 can have any shape or size, including, but not limited to, substantially rectangular, substantially square, substantially round, substantially oval, substantially ellipsoid, a butterfly bandage shape, or any other regular or irregular shape. In some embodiments, sharp corners of the fabric 111 are rounded to reduce the possibility of the tape 100 curling away from the body at the corners. In at least one embodiment, the tape 100 can be pre-cut and have an unstretched length greater than its unstretched width, as shown. In other embodiments, the tape 100 is provided in bulk as a roll of tape, such as shown in FIG. 2, which can be cut to length.

The tape 100 includes a heating element 113 coupled to the fabric material 111. In some embodiments, the heating element 113 is positioned on the second side of the fabric material 111 as shown in FIG. 1B. The heating element 113 can be positioned on the fabric material using conductive ink applied to the fabric material 111, which may be applied using a printing process, or by affixing conductive fibers to the fabric material 111. In other embodiments, the heating element 113 can be formed with conductive fibers woven into the fabric material 111. In yet other embodiments, the heating element 113 may be positioned between the pressure sensitive adhesive 119 and the fabric material 111. The pressure sensitive adhesive 119 may have electrical insulating properties to isolate the skin of the patient from any electrical energy applied to the heating element 113. In at least one embodiment, the heating element 113 may be created in the pressure sensitive adhesive 119 by making the adhesive 119 itself conductive or by adding conductive material to the adhesive 119.

The tape 100 also includes electrical contacts 115A/B electrically coupled to the heating element 113 and accessible from the second side of the fabric material 111, the non-skin facing side of the tape 100. In some embodiments, the electrical contacts 115A/B may simply be exposed areas of the heating element 113. In some embodiments, a low resistance conductor may be included on or in the tape to electrically connect the heating element 113 to an electrical contact 115A/B. In various other embodiments, the electrical contacts may be formed using conductive loop fasteners, conductive hook fasteners, conductive snap fasteners, or conductive surfaces adapted for use with conductive glue. Any method may be used to electrically couple the electrical contact with the heating element 113 including, but not limited to, sewing with conductive thread, attaching with conductive glue, weaving into the fabric tape, or any other method to keep the electrical contacts 115A/B electrically coupled to the heating element 113. The tape 100 can have two or more electrical contacts but include a first electrical contact 115A and a second electrical contact 115B to allow the heating element 113 to be electrically powered through the electrical contacts 115A/B. In various embodiments, the two electrical contacts 115A/B can have any special relationship with each other, including being on opposite ends of the heating element 113, being positioned at the same end of the heating element 113 with a low resistance conductor electrically connecting one of the electrical contacts to the opposite end of the heating element 113, positioned near each other at the ends of with a ‘U-shaped’ heating element, or any other spatial relationship.

FIG. 1C shows the non-skin-facing side of an embodiment of a heating kinesiology tape 100S in a stretched position. In the embodiment shown, the anisotropically stretchable fabric 1115 has its stretchable direction aligned with the length of the tape 100/100S. The piece of anisotropically stretchable fabric 111/100S in this embodiment is stretchable to at least 150% of a length of the fabric in a direction of the length, and stretchable to no more than 110% of a width of the fabric in a direction of the width. In some embodiments, the tape 100 is stretchable to 180% of its length, so after being stretched, the tape 100S of FIG. 1B has a length that is 80% longer, as shown in FIG. 1C.

In some embodiments, the heating element 113 is formed as a serpentine shaped resistive element 113 extending longitudinally along the adhesive tape 100 as shown in FIG. 1B/C. The serpentine shape allows the tape 100 to stretch between 100% of its original length and as much as 180% of its original length without putting much tensile stress on the resistive element 113. Other embodiments may use other shapes for the heating element 113, such as a circular shape, a fractal shape, a wide ribbon, or a woven heating element. Any shape may be used as long as the heating element 113 maintains electrical continuity as the heating kinesiology tape 100 is stretched to be stretched heating kinesiology tape 100S. In embodiments, the serpentine shaped resistive element 113 retains electrical continuity when the adhesive tape 100 is stretched to 180% of its original length. The serpentine shaped electrical element can be formed inside of the adhesive 119, inside of the fabric material 111, or on either side of the fabric material 111, depending on the embodiment. The total resistance of the serpentine shaped heating element 113 may not vary significantly as it is stretched because the actual length of the heating element 113 does not significantly change as it is stretched; the spacing of the serpentine segments is simply increased. In other embodiments, which may or may not be serpentine, the resistance of the heating element may or may not change, depending on the shape and mechanical structure of the embodiment of the heating element.

FIG. 2 shows a roll of an embodiment of heating kinesiology tape 200. The roll can be of any length. The heating kinesiology tape 200 includes an anisotropically stretchable fabric tape 211 with adhesive applied to a skin-facing side of the fabric tape 211 and a longitudinal heating element 213. The heating element 213 may be formed in the adhesive, in the fabric tape 211 or on either or both sides of the fabric tape 211. The heating element 213 may be formed using any suitable material and/or method.

In some embodiments, the heating element 213 may be formed using conductive yarn that may be woven into the fabric tape 211 or affixed to a surface of the fabric tape 211. The conductive yarn may be formed into a serpentine shaped heating element 213 in some embodiments. The conductive yarn may be formed around a core of insulating material with an electrical resistance heating filament disposed generally about the core. A sheath material may be included that generally surrounds the electrical resistance heating filament and the core. The core can be a yarn of synthetic material, such as polyester, or a natural material such as cotton, or any other suitable yarn. The sheath material may be made from the same yarn as the core or may be a different type of yarn. In some embodiments of the conductive yarn, the core, and/or the sheath material may be omitted. In some embodiments, the electrical resistance heating filament includes one or more metal filaments wrapped helically about the core. The metal filaments may be made of any type of metallic material with a suitable resistance, such as stainless steel. In other embodiments, the electrical resistance heating filament includes one or more non-metallic filaments wrapped helically about the core, such as carbon fibers. In some embodiments, the conductive yarn has electrical resistance in the range of about 0.1 ohm/cm to about 100 ohm/cm. Depending on the embodiment, the heating element 213 may be formed with a single piece of conductive yarn or with 2 or more pieces of conductive yard in parallel.

In some embodiments, the heating element 213 may be formed using conductive fibers that may be embedded in the adhesive or woven into the fabric tape 211. Any suitable conductive, heat-generating fiber can be used, including, but not limited to, metallic strands, non-conductive fibers coated with conductive materials, non-conductive fibers impregnated with conductive materials, or carbon fibers. Carbon fibers made from a polyacrylonitrile precursor may be used in some embodiments. Commercially available carbon fibers include FORTAFIL® carbon fiber from Fortafil Fibers, Inc. of Knoxville, Tenn., SIGRAFIL® C carbon fibers from SGL Carbon AG of Wiesbaden, Germany, and Thornel® carbon fibers from CYTEC Industries, Inc. of West Paterson, N.J. Other embodiments may utilize carbon or metal-based powders incorporated into the adhesive or impregnating the fabric tape 211 to create the heating element 213.

In some embodiments, the heating element 213 may be formed using conductive ink. The conductive ink may be printed onto the fabric tape 211. Screen printing technology may be used in some embodiments. In some embodiments, a serpentine pattern may be printed onto the fabric tape 211 while it is stretched to its maximum intended stretching point, such as 180% in some embodiments, 150% in other embodiments, or any other stretch amount based on the properties of the anisotropically stretchable fabric tape 211. In other embodiments, other patterns may be used as long as electrical conductivity can be maintained through the heating element 213 throughout the range of stretch of the fabric tape from its unstretched state to its maximum stretch state. Any type of conductive ink may be used depending on the embodiment, such as, but not limited to, polymer thick film materials, carbon-based resistive paste, and silver-based conductive inks. Conductive inks can be obtained from a variety of sources including Thermo Heating Elements, LLC of Piedmont, S.C., and DuPont of Wilmington, Del.

The heating kinesiology tape 200 includes electrical contacts, 215A/B/C. The roll of tape 200 may include any number of electrical contacts, such as 3 or more electrical contacts which may be distributed substantially equally along the adhesive tape longitudinally. The electrical contacts may be spaced along the unstretched adhesive tape substantially at a predetermined distance 217 apart. The distance 217 between contacts may vary according to the embodiment and can be any distance in the range of about 1 centimeter (cm) to about 1 meter. In many embodiments, the distance 217 between contacts may be between about 5 cm and about 50 cm. At least one embodiment has contacts spaced at a distance 217 of about 15 cm. In some embodiments, the heating kinesiology tape may be precut into predetermined lengths and may include only two electrical contacts positioned near the ends of the precut pieces of tape, so that the distance between the two electrical contacts is determined by the length of the precut piece of tape. In other embodiments, such as the roll of heating kinesiology tape 200, the electrical contacts are spaced closely enough to allow a variety of useful lengths of tape to be cut and still have one electrical contact close to one end and another electrical contact close to the other end.

FIG. 3 shows an embodiment of heating kinesiology tape system used to treat 300 a muscle on a human leg 301. The heating kinesiology tape 310 is applied to the leg 301 by stretching the heating kinesiology tape 310 to at least 125% of its unstretched length, and positioning the stretched heating kinesiology tape 310 on the leg 301 at an appropriate place to treat 300 the muscle. The stretched heating kinesiology tape 310 is then pressed against the leg 301 to activate a pressure-sensitive adhesive on the heating kinesiology tape 310 and adhere the stretched heating kinesiology tape 310 to the leg 301.

The heating kinesiology tape 310, which is an adhesive tape, can be a part of a heating tape system that also includes a power supply 320. The heating kinesiology tape 310 includes an anisotropically stretchable fabric material 311 having a first side and a second side opposite the first side. A pressure sensitive adhesive is fixed on the first side, or skin-facing side, of the fabric material 311 and a heating element 313 is coupled to the fabric material 311. A first load contact 315A and a second load contact 315D are both electrically coupled to the heating element 313 and are accessible from the second side, or non-skin-facing side, of the fabric material 311. Depending on the embodiment and the length of the heating kinesiology tape 310, additional contacts, such as third contact 315B and fourth contact 315C, may also be electrically coupled to the heating element 313 and are accessible from the second side of the fabric material 311.

The power supply 320 can include a case 321 that may enclose a source of electrical energy with a first terminal and a second terminal. The power supply 320 also includes a first source contact 325 electrically coupled to the first terminal of the source of electrical energy and adapted to be electrically coupled to the first load contact 315A. In the embodiment shown, the power supply 320 includes a wire 324 electrically coupling the source of electrical energy to the first source contact 325. The power supply 320 also includes a second source contact 327 electrically coupled to the second terminal of the source of electrical energy and adapted to be electrically coupled to the second load contact 315D. In the embodiment shown, the conductor 326 electrically couples the second source contact 327 to the second terminal of the source of electrical energy in the case 321. In some embodiments, the combined length of the wire 324, the case 321, and the conductor 326 is based on the distance between two selected contacts 315A/D on the stretched tape 310, and the voltage level of the source of electrical energy in the case 321 is based on the resistance of the heating element 313 between the two selected contacts 315A/D. Any method can be used to electrically couple the source contacts to the load contacts, but in at least one embodiment, the system also includes conductive glue to electrically couple the first source contact 325 to the first load contact 315A and the second source contact 327 to the second load contact 315D. In another embodiment, conductive adhesive is affixed to the first source contact 325 and the second source contact 327. The conductive adhesive is adapted to electrically couple the source contacts 325/327 to the load contacts 315A/D. The adhesive on the source contacts 325/327 may be covered with a release paper to protect the adhesive until the heating tape system is applied. Heat is generated by the heating element 313 when an electrical voltage is applied between the first load contact 315A and the second electrical contact 315D by the power supply 320.

In some embodiments, the adhesive tape 310 also includes a power supply attachment area with loop fasteners located at a first distance from the first load contact 315A on the unstretched adhesive tape 310 and conductive loop fasteners on the first load contact 315A. In at least some embodiments, the power supply attachment area is located between the first load contact 315A and the second load contact 315D. In such embodiments, the power supply also includes hook fasteners on the case 321 to mate with the loop fasteners of the power supply attachment space on the adhesive tape 310. In other embodiments, the adhesive tape 310 may have hook fasteners in the power supply attachment area and the power supply case 321 may have loop fasteners to mate with the hook fasteners on the adhesive tape. In some embodiments the power supply also has conductive hook fasteners on the first source contact 325 to mate with the conductive loop fasteners of the first load contact 315A and a conductor 324 extending from the case 321 electrically coupling the source of electrical energy to the first source contact 325. The conductor 324 can have a length based on the first distance, which is the distance from the power supply attachment area to the first load contact 315A, and a stretch factor for the adhesive tape 310. For example, if the distance from the power supply attachment area to the first load contact 315A is 30 cm on the unstretched tape 310 and the tape 310 is to be stretched to 150% of its original length before application to the leg 301, the length of the conductor 324 may be about 30×1.5=45 cm. In this example, the conductor 324 is long enough to allow for attachment of the first source contact 325 to the first load contact 315A using the conductive hook and loop fasteners while the case 321 of the power supply 320 is attached to the power supply attachment area using the hook and loop fasteners. The conductor 324 then has a little bit of slack, based on the size of the case 321, to allow for easy attachment of the first source contact 325 to the first load contact 315A.

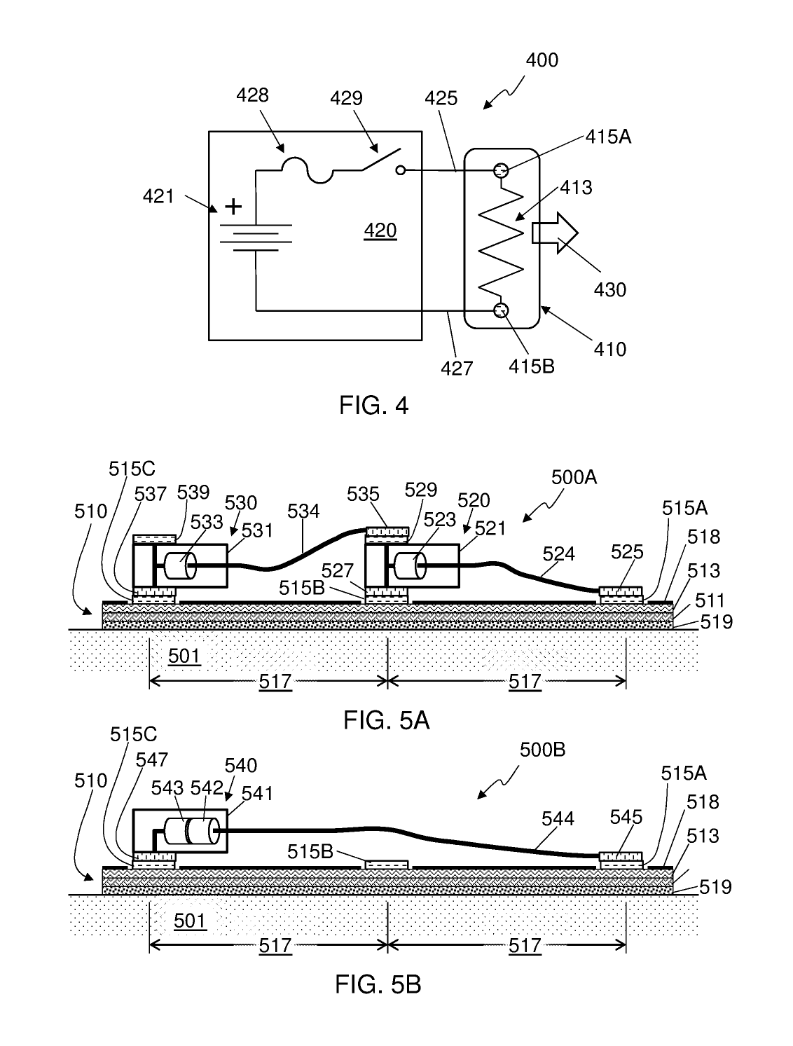

FIG. 4 is a schematic view 400 of an embodiment of heating kinesiology tape 410 with a power source 420. The heating kinesiology tape 410 includes a heating element 413, a first load contact 415A, and a second load contact 415B. The first load contact 415A is electrically coupled to the heating element 413 near one end of the heating kinesiology tape 410, and the second load contact 415B is electrically coupled to the heating element 413 near the other end of the heating kinesiology tape 410.

The power supply 420 includes a source of electrical energy 421. The source of electrical energy may provide direct current (DC) or alternating current (AC), depending on the embodiment. In some embodiments, the source of electrical energy 421 may include a battery and/or a battery holder. The source of electrical energy 421 may be a fixed voltage source, such as a voltage regulated source. For the purposes of this disclosure and the claims, a battery is also considered a fixed voltage source even though the voltage of the battery may vary over load and over time as the energy of the battery is depleted. In some embodiments, the source of electrical energy 421 may be a fixed current source. For the purposes of this disclosure and the claims, if the source of electrical energy 421 regulates its output based on the current, it is considered a fixed current source even if the current may vary somewhat over load and energy level of the source of electrical energy 421. The source of electrical energy can incorporate any type of electrical energy source including, but not limited to, a battery, a fuel cell, a solar cell, a kinetic energy based generator, a motor based generator, a wind generator, or plug-in power cord, and may incorporate any type of circuitry including, but not limited to, transformers, rectifiers, capacitors, transistors, voltage based regulators, current based regulators, or power based regulators. In some embodiments, a temperature sensor may be included in the heating kinesiology tape and/or the power supply which can be used to control the power supply, so some embodiments may incorporate circuitry for temperature based regulation of the power supply.

The power supply 420 also includes a first source contact 425 to electrically couple a first terminal of the source of electrical energy 421 to the first load contact 415A and a second source contact 427 to electrically couple a second terminal of the source of electrical energy 421 to the second load contact 415B. In some embodiments, the power supply 420 also includes a fuse 428 coupled between the source of electrical energy 421 and the first source contact 425. In some embodiments, the energy level of the source of electrical energy 421 is low enough that the fuse 428 may be eliminated. In some embodiments, the power supply 420 also includes an on/off switch 429 coupled between the source of electrical energy 421 and the first source contact 425. Other embodiments eliminate the on/off switch and simply provide power to the heating kinesiology tape upon the electrical coupling of the source contacts 425/427 to the load contacts 415A/B.

When power is provided to the heating kinesiology tape 410, heat 430 is generated by the heating element 413. The heating element 413 may be designed to evenly spread the heat over the area of the heating kinesiology tape 410. An amount of power may be provided to the heating element 413 to maintain a target temperature. The power needed to attain the target temperature is impacted by several factors and may depend on the specific thermal properties of the fabric tape and adhesive used in the heating kinesiology tape. The target temperature may vary, depending on the therapeutic goals. In some embodiments, the target temperature may be any temperature between about 25° C. and 40° C. While the typical human core body temperature is 37° C., human skin temperature can vary greatly depending on the ambient temperature, the part of the body, and the current activity level. Sweat evaporation, which is based on a variety of factors including humidity levels and air movement, can also greatly impact skin temperature. But a reasonable assumption for a skin temperature of a portion of the human body that is likely to be treated with kinesiology tape, within an ambient temperature range of about 23° C. to about 33° C., is about 30° C. to about 35° C. So in many cases, a target temperature for the heating kinesiology tape may be above 35° C. In some embodiments, a target temperature range of between the human core body temperature of 37° C. and about 40° C. may be appropriate, and in at least one embodiment, a target temperature of about 39° C. is used.

Some embodiments may not generate enough heat to be felt by the person that the heating kinesiology tape is applied to. In some cases, very small amounts of heat, such as 500 milliWatts (mW) or less may be dissipated over the entire piece of heating kinesiology tape. In some cases, as little as 50 mW may be provided to a piece of heating kinesiology tape having an area of as much as 100 cm² for a power density as low as 0.5 mW/cm². At a 50 mW power level, a single AA battery may be able to provide power to the heating kinesiology tape for several days. At a 500 mW power level, two AA batteries may be able to provide power to the heating kinesiology tape for over 12 hours.

In embodiments, the heating element 413 is formed in such a way that it has a resistance based on the length of the heating kinesiology tape 410. Various lengths of heating kinesiology tape may be cut from a bulk source, such as the roll of heating kinesiology tape 200 of FIG. 2. Different lengths of heating kinesiology tape have different resistances. In embodiments where the electrical contacts are spaced at regular intervals, the resistance between any two adjacent electrical contacts may have about the same value, a section resistance ‘Rs’. The section resistance may or may not change as the tape is stretched, depending on the embodiment. The resistance between any two electrical contacts on the same piece of heating kinesiology tape can then be simply calculated by counting the number of electrical contacts located on the piece of heating kinesiology tape, subtracting ‘1’ from that number and multiplying by ‘Rs’ to obtain the total resistance of the piece of heating kinesiology tape ‘Rt’.

The amount of heat 430 generated by the piece of heating kinesiology tape 410 is based on the resistance of the heating kinesiology tape 410 and the electrical characteristics of the power supply 410. For the purposes of this example, it is assumed that the first load contact 415A and the second load contact 415B are the end contacts on the piece of heating kinesiology tape 410 so the resistance of the heating kinesiology tape 410 is the total resistance of the piece of heating kinesiology tape ‘Rt’. Power can be calculated based on current and resistance as I²×R, so if a constant current source is used, the power dissipated by a piece of tape is directly proportional to its length, which provides a constant amount of heat per unit area. If the power supply 420 is a constant current source, the therapeutic effect of the heat would be consistent, independent of the length of the piece of heating kinesiology tape 410. Constant current sources, however, require relatively complex circuitry as traditional portable power sources, such as batteries, and fuel cells, are not constant current sources. This makes constant current sources more expensive, in general, than a constant voltage source of equivalent power delivery capability.

Power can also be calculated based on voltage and resistance as V²/R, so if a constant voltage source such as a battery or a fuel cell is used, the total power dissipated by the piece of tape goes down linearly with the length of the tape which means the heat per unit area actually goes down with the square of the length. So, a single constant voltage source power supply, which generates an appropriate amount of heat when used with a first piece of heating kinesiology tape with a given length, may not generate enough heat for the desired therapeutic effect when used with a second piece of heating kinesiology tape that is twice as long. In this example, the second piece of heating kinesiology tape would only generate one quarter of the heat per unit area as the first piece of heating kinesiology tape.

One way to provide a constant therapeutic effect is to provide a plurality of power supplies that are individually matched to different lengths of tape. So for example a power supply that is matched for a piece of heating kinesiology tape of length L1, i.e. about the distance between adjacent electrical contacts on the heating kinesiology tape, may have a voltage of V1 to generate an amount of heat per unit area of H. A second power supply that is matched for a piece of heating kinesiology tape of length L2 which is twice as long as L1 (i.e. a piece of heating kinesiology tape with three electrical contacts) may have a voltage of V2 which is twice the voltage V1 to generate the same amount of heat per unit area of H. A third power supply that is matched for a piece of heating kinesiology tape of length L3 which is three times as long as L1 (i.e. a piece of heating kinesiology tape with four electrical contacts) may have a voltage of V3 which is three times the voltage V1 to generate the same amount of heat per unit area of H. Any number of different power supplies matched to different lengths of tape can be provided. In some embodiments, each type of power supply may be fitted with leads of the appropriate length to match the length of its corresponding tape. So in some embodiments, the power supply 420 includes a fixed voltage source 421 having a voltage based on a targeted length of the adhesive tape 410, and a length of the wire electrically coupling the source of electrical energy 421 to the first source contact 425 is based on the targeted length of the adhesive tape 410, where the adhesive tape 410 is heating kinesiology tape.

In another embodiment, different levels of heat output may be desired so different power supplies with different power delivery characteristics may be provided for a particular length of heating kinesiology tape. The different power supplies may have the same length of wire, indicating that they are targeted for use with the same length of heating kinesiology tape, but may be marked to indicate different power levels. The markings on the power supplies may include one or more of words, phrases, icons, pictures, or color coding.

In one example, 10 mW/cm² is desired for a particular therapeutic effect in using a piece of heating kinesiology tape that is 2 cm wide and has an unstretched length of 12 cm with load contacts located very close to each end. The heating kinesiology tape of this example has a recommended stretch of 150%, providing a stretched length of 18 cm for the piece of tape and a stretched area of 36 cm². This means that a total power (Pt) of 360 mW is required for the heated kinesiology tape to hit its target of 10 mW/cm². If a single alkaline AA battery is targeted for use in the power supply with a nominal voltage of 1.5 V, a target Rt for the tape can be calculated using V²/Pt as 6.25 ohms (Q). A battery having a capacity of 2600 milliamp-hours (mAh) could potentially power the piece of heating kinesiology tape at the 10 mW/cm² for as long as about 10 hours. Because batteries do not provide a constant voltage over their entire lifetime at any possible current, a more accurate, and more complex, calculation of a target resistance and battery life based on the discharge characteristics of the battery may be used in some embodiments.

FIG. 5A shows a cross-sectional view of an embodiment of a heating tape system 500A. In embodiments, a heating tape system may include one or more substantially identical power supplies, along with one or more pre-cut pieces of heating kinesiology tape and/or one or more bulk rolls of heating kinesiology tape. The heating tape system 500A includes a piece of kinesiology tape 510 powered by a first power supply 520 and a second power supply 530. The piece of heating kinesiology tape 510 is adhered to a human body 501 in a stretched state. The piece of heating kinesiology tape 510, which is an adhesive tape, includes an anisotropically stretchable piece of fabric material 511 with a pressure sensitive adhesive 519 on the first (skin-facing) side of the fabric material 511. A heating element 513 is shown on the second (non-skin-facing) side of the fabric material 511, but other embodiments may include the heating element 513 in the adhesive material 519, between the adhesive material 519 and the fabric material 511, or in the fabric material 511. The adhesive tape 510 includes two or more electrical contacts electrically coupled to the heating element 513, such as a first load contact 515A, a second load contact 515B, and a third load contact 515C. In some embodiments, an insulating layer 518 may be affixed to the second side of the fabric material 511, covering the heating element 513. In some embodiments, the insulating layer 518 may have openings to enable the electrical load contacts 515A/B/C to be electrically coupled to the heating element 513.

The load contacts 515A/B/C can be spaced a fixed distance 517 apart on the heating kinesiology tape 510. In the embodiment shown, the first load contact 515A includes conductive loop fasteners and is located at a distance 517 from the second load contact 515B which also includes conductive loop fasteners. The third load contact 515C, which also includes conducting loop fasteners, is located at the first distance 517 from the second load contact 515B opposite from the first load contact 515A. In other embodiments, the load contacts 515A/B/C may be made of any conductive material and can be electrically coupled to the heating element 513 by any suitable method. In some embodiments, the load contacts 515A/B/C include conductive hook fasteners, conductive snaps, or conductive areas adapted for use with conductive glue.

In some embodiments, the heating kinesiology tape 510 may have a power supply attachment area adapted to have the power supply attached. In some embodiments, the tape 510 may include a power supply attachment area between each pair of load contacts, such as between the first load contact 515A and the second load contact 515B, and between the second load contact 515B and the third load contact 515C. In some embodiments, the power supply attachment areas include loop or hook fasteners to mate with hook or loop fasteners on the power supply. In some embodiments, a load contact can act as the power supply attachment area and conductive loop fasteners may be included in the power supply attachment area to act as the load contact, such as the second load contact 515B and the third load contact 515C.

The first power supply 520 may be used to provide power to a first portion of the heating kinesiology tape 510. The first power supply 520 includes a case 521 to enclose a source of electrical energy, such as a single battery 523 (which may include multiple cells in some embodiments). Other embodiments may utilize a different type of a source of electrical energy. Various battery-based embodiments may utilize different numbers of batteries, different battery chemistries and/or different battery form factors. Embodiments may utilize a single AA alkaline battery, a single AAA alkaline or rechargeable battery, a single 9V alkaline battery, a Li-Ion battery with a custom form factor, a CR2032 lithium battery, a thin film battery, or any other type of battery in any configuration. In at least one embodiment, a thin-film battery may be affixed to the tape 510 and provided with the tape 510.

The first power supply 520 includes a first source contact 525 that is coupled to a first terminal of the battery 523, such as the anode of the battery 523, by a wire 524, and a second source contact 527 that is coupled to a second terminal of the battery 523, such as the cathode of the battery 523. In the embodiment shown, the first source contact 525 includes conductive hook fasteners to mate with the conductive loop fasteners of the first load contact 515A, and conductive hook fasteners are provided on the case to act as the second source contact 527 to mate with the conductive loop fasteners of the second load contact 515B. In some embodiments, the first power supply 520 also includes additional conductive loop fasteners, on a different side of the case 520 from the second source contact 527, to act as a daisy-chain contact 529. The daisy-chain contact 529 is electrically coupled to the second source contact 527.

The second power supply 530 may be used to provide power to a second portion of the heating kinesiology tape 510. In the system shown, the second power supply 530 is substantially the same as the first power supply 520. The second power supply 530 includes a case 531 to enclose a first source of electrical energy, such as a single battery 533. The second power supply 530 includes a first source contact 535 that is coupled to a first terminal of the battery 533 by a wire 534 and a second source contact 537 that is coupled to a second terminal of the battery 533. In the embodiment shown, the conductive hook fasteners on the first source contact 535 of the second power supply 530 are adapted to mate with the conductive loop fasteners of the daisy-chain contact 529 of the first power supply 520. Conductive hook fasteners are provided on the case 531 of the second power supply 530 to act as the second source contact 537 of the second power supply and are adapted to mate with the conductive loop fasteners of the third load contact 515C. In some embodiments, the second power supply 530 also includes additional conductive loop fasteners, on a different side of the case 531 from the second source contact 537, to act as a daisy-chain contact 539 which is electrically coupled to the second source contact 537.

A first example heating tape system includes a bulk roll of heating kinesiology tape having load contacts spaced at 15 cm intervals that may be cut into lengths of about 16 cm to about 44 cm with two load contacts, lengths of about 31 cm to about 59 cm with three load contacts, or lengths of about 46 cm to about 74 cm with four load contacts, where the lengths are measured with the tape unstretched. The example heating kinesiology tape is recommended for application while stretched to about 133% of its unstretched length. The load contacts of the heating kinesiology tape are made of conductive loop fasteners.

The first example heating tape system includes one or more substantially identical power supplies. The power supply of the first example heating tape system has a battery holder for a single AA battery and a wire about 20 cm (˜1×15 cm×133%) long coupling the anode of the battery holder to a first source contact with conductive hook fasteners. The power supply has a second source contact with conductive hook fasteners on one side of the power supply case and a daisy-chain contact with conductive loop fasteners on the opposite side of the power supply. Both the second source contact and the daisy-chain contact are electrically coupled to the cathode of the battery holder.

At a time of treatment of the human, a piece of heating kinesiology tape of appropriate length to treat the portion of the human body that has been identified for treatment is cut from the bulk roll. The piece is stretched and applied to the identified portion of the human body. Then an appropriate number of power supplies are selected. One way of selecting the proper number of power supplies is to count the number of contacts on the piece of tape, subtract one, and select that number of the power supplies. Once the power supplies have been selected, their battery holders can be populated with AA batteries. A first power supply can then be attached near one end of the tape and its second source contact electrically coupled to a load contact of the tape near that end of the tape. The first power supply may be held in place by the conductive hook and loop fasteners of the contacts. A second power supply can then be attached to the next load contact, electrically coupling that load contact to the power supply's second source contact. The second power supply may be held in place by the conductive hook and loop fasteners of the contacts. The first source contact of the first power supply can then be attached to the daisy-chain contact of the second power supply. This process is repeated until only one load contact is still exposed on the piece of tape. Then the first source contact of the final power supply is attached to the final load contact near the opposite end of the tape. In the first example heating tape system, a separate power supply is used to power each section of the heating kinesiology tape located between adjacent load contacts.

FIG. 5B shows a cross-sectional view of an alternative embodiment of a heating tape system 500B. The heating tape system 500B includes the same piece of heating kinesiology tape 510 of FIG. 5A, but is powered by a single power supply 540. In embodiments, a heating tape system may include one or more pre-cut pieces of heating kinesiology tape and/or a bulk roll of heating kinesiology tape, and one or more power supplies of two or more types. As in FIG. 5A, the piece of heating kinesiology tape 510 is adhered to a human body 501 in a stretched state. The piece of heating kinesiology tape 510, which is an adhesive tape, includes an anisotropically stretchable piece of fabric material 511 with a pressure sensitive adhesive 519 on the first (skin-facing) side of the fabric material 511. The adhesive tape 510 includes two or more electrical contacts electrically coupled to the heating element 513, such as a first load contact 515A, a second load contact 515B, and a third load contact 515C. In some embodiments, an insulating layer 518 may be affixed to the second side of the fabric material 511 and covering the heating element 513, the insulating layer 519 having openings to expose the electrical load contacts 515A/B/C.

The load contacts 515A/B/C can be spaced a fixed distance 517 apart on the heating kinesiology tape 510. In the embodiment shown, the first load contact 515A includes conductive loop fasteners and is located at a distance 517 from the second load contact 515B which also includes conductive loop fasteners. The third load contact 515C, which also includes conducting loop fasteners, is located at the first distance 517 from the second load contact 515B opposite from the first load contact 515A so that the third load contact 515C is twice the first distance 517 from the first load contact 515A.

In FIG. 5B a third power supply 540 may be used to provide power to the entire piece of heating kinesiology tape 510. The third power supply 540 includes a case 541 to enclose a source of electrical energy, such as two batteries 542, 543 connected in series with the cathode of the first battery 542 electrically coupled to the anode of the second battery 543. The third power supply 540 includes a first source contact 545 that is coupled to the anode of the first battery 542 by a wire 544, and a second source contact 547 that is coupled to the cathode of the second battery 543. In the embodiment shown, the first source contact 545 includes conductive hook fasteners to mate with the conductive loop fasteners of the first load contact 515A, and conductive hook fasteners are provided on the case 541 to act as the second source contact 547 to mate with the conductive loop fasteners of the second load contact 515B.

In the embodiment shown, the wire 544 of the third power supply 530 has a length about twice the length of the wire 524 of the first power supply 520. The source of electrical energy of the first power supply 520 utilizes a single battery, and the source of electrical energy of the third power supply 540 utilizes two batteries in series that are the same type used in the first power supply 520. Because of this, the source of electrical energy of the first power supply 520 has a first voltage based on the voltage of the battery 523, and the source of electrical energy of the third power supply has a second voltage about twice that of the first voltage.

A second example heating tape system includes a bulk roll of heating kinesiology tape having load contacts spaced at 10 cm intervals that may be cut into lengths of about 9 cm to about 21 cm with two load contacts, lengths of about 19 cm to about 31 cm with three load contacts, or lengths of about 29 cm to about 41 cm with four load contacts, where the lengths are measured with the tape unstretched. The example heating kinesiology tape is recommended for application while stretched to about 150% of its unstretched length. The load contacts of the heating kinesiology tape are made of conductive loop fasteners. The second example heating tape system includes one or more of each of three types of power supplies. The first type of power supply is marked with two dots and has a battery holder for a single AA battery and a wire about 15 cm (˜1×10 cm×150%) long coupling the anode of the battery holder to a first source contact with conductive hook fasteners. The second type of power supply is marked with three dots and has a battery holder for two AA batteries connected in series and a wire about 30 cm (˜2×10 cm×150%) long coupling the anode of the battery holder to a first source contact with conductive hook fasteners. The third type of power supply is marked with four dots and has a battery holder for three AA batteries connected in series and a wire about 45 cm (˜3×10 cm×150%) long coupling the anode of the battery holder to a first source contact with conductive hook fasteners. Each type of power supply has a second source contact with conductive hook fasteners on one side of the power supply case that is electrically coupled to the cathode of the battery holder.

At a time of treatment of the human, a piece of heating kinesiology tape of appropriate length to treat the portion of the human body that has been identified for treatment is cut from the bulk roll. The piece is stretched and applied to the identified portion of the human body. Then an appropriate power supply is selected. One way of selecting a power supply is to select the power supply with the shortest wire that can be coupled to the load contacts that are closest to the ends of the piece of tape. Another way of selecting the power supply is to count the number of contacts on the piece of tape and select the power supply with that number of dots. Once the appropriate power supply has been selected, its battery holder can be populated with AA batteries and the power supply attached near one end of the tape with its second source contact electrically coupled to a load contact of the tape and held in place by the conductive hook and loop fasteners of the contacts. The first source contact can then be affixed to the load contact close to the other end of the tape via the conductive hook and loop fasteners of those contacts.

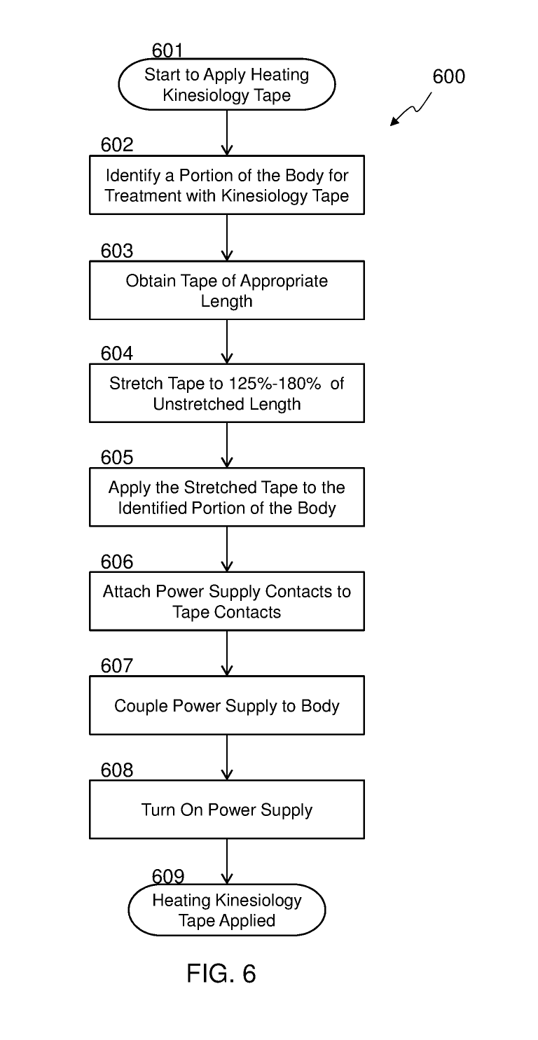

FIG. 6 is a flowchart 600 of an embodiment of a method of applying heating kinesiology tape to a body. The flowchart 600 begins at block 601 by starting to apply heating kinesiology tape to a body. A portion of a human body is identified for treatment with heating kinesiology tape at block 602. Any method may be used to identify the portion of the human body for treatment including, but not limited to, inspection of the human body, patient description of pain, X-Rays, CT Scans, manual manipulation of the human body, or thermal imaging. Once the portion of the human body has been identified for treatment, a piece of heating kinesiology tape of an appropriate length for treating the identified portion of the human body is obtained at block 603. The piece of heating kinesiology tape includes an anisotropically stretchable fabric material, a pressure sensitive adhesive fixed to the fabric material, a heating element coupled to the fabric material, and a first load contact and a second load contact, both electrically coupled to the heating element and positioned a distance apart. The piece of heating kinesiology tape of the appropriate length may be obtained by selecting a pre-cut length of kinesiology tape, or by cutting a piece of heating kinesiology tape of the appropriate length from a bulk roll or a longer piece of heating kinesiology tape. The stretch factor of the heating kinesiology tape may be taken into account in determining the appropriate length because the piece of heating kinesiology tape will be applied in a stretched state. Care may also be taken to ensure that the first load contact is located near one end of the piece of heating kinesiology tape and the second load contact is located near the opposite end of the piece of heating kinesiology tape. The length of the piece of heating kinesiology tape may be based, in part, on the spacing of the electrical contacts on the heating kinesiology tape, either rounded up or rounded down from the initially determined length, to ensure that the load contacts are located near the ends of the piece of tape.

In at least one embodiment, the load contacts are provided separately from the fabric material and adapted to be electrically coupled to the heating element by an individual at a time of application of the adhesive tape to a human body. The load contacts may then be installed on the tape near the ends of the tape to be electrically coupled to the heating element. Various mechanisms can be used to allow the load contacts to be user installed, including, press-fit conductive rivets, a first device with one or more sharpened metallic prongs adapted to be pushed through the tape from the adhesive side into a contact positioned on the opposite side of the tape, or any other mechanism to allow a user to affix the contacts to the tape and electrically couple the contacts to the heating element.

The method of applying the heating kinesiology tape includes stretching the piece of heating kinesiology tape to at least 125% of its unstretched length at block 604. In some embodiments, the heating kinesiology tape is stretched to between 130% and 175% of its unstretched length. In at least one embodiment, the heating kinesiology tape is stretched to about 180% of its unstretched length. At block 605, the stretched heating kinesiology tape is applied to the identified portion of the human body. In at least one embodiment, the stretched heating kinesiology tape is positioned with one end of the piece of heating kinesiology tape at the origin of a muscle in the identified portion of the human body, or where the muscle is attached to an immovable bone. An opposite end of the piece of heating kinesiology tape is positioned at the insertion point of the muscle, or where the muscle is attached to a movable bone.

In some embodiments, the method includes selecting the power supply from a plurality of power supplies based on a stretched length of the piece of heating kinesiology tape. The selecting may also be based on a length of a conductor coupling the first source contact to the selected power supply.

The flowchart 600 continues at block 606 with attaching a first source contact of a power supply to the first load contact and attaching a second source contact of the power supply to the second load contact to create an electrical circuit with the power supply and the heating element. In some embodiments, the power supply is coupled to the human body at block 607. This may be done by using hook and loop fasteners on the tape and power supply, gluing the power supply to the tape, using other tape to secure the power supply to the human body, putting the power supply in a pocket of clothing, or by any other appropriate method. Some power supplies may include a power switch, which may be turned on in block 608 to provide electrical current from the power supply to the heating element. In embodiments where the power supply does not have a switch, electrical current may begin to flow from the power supply to the heating element once both source contacts are connected to their respective load contacts. The heating kinesiology tape has been successfully applied at block 609.

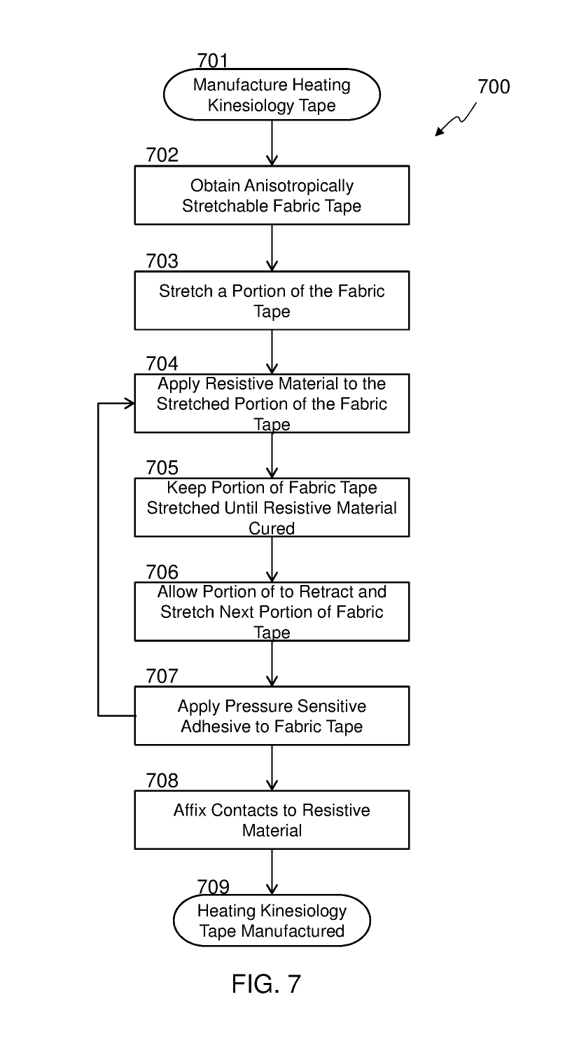

FIG. 7 is a flowchart 700 of an embodiment of a method of manufacturing heating kinesiology tape. The flowchart 700 begins at block 701 by starting to manufacture heating kinesiology tape. Anisotropically stretchable fabric tape is obtained at block 702. The fabric tape may be obtained by weaving fibers into a fabric tape, knitting fibers into a fabric tape, purchasing the fabric tape, or any other method. The fabric tape is longitudinally stretchable. The method continues at block 703 by stretching a portion of the fabric tape longitudinally. Resistive material is applied to the stretched portion of the fabric tape at block 704. In some embodiments, the method includes keeping the stretched portion of the fabric stretched for a curing time of the resistive material at block 705. The method continues with unstretching the stretched portion of the fabric tape at block 706. Pressure sensitive adhesive is applied to the fabric tape at block 707. In some embodiments, the method repeats block 704, block 705, block 706, and block 707 with successive portions of the fabric tape in a continuous process. In some embodiments, the method also includes applying an insulating layer on top of the resistive material with a gap in the insulating layer at a regular interval to expose the resistive material.

The flowchart 700 continues with block 708 in some embodiments with affixing contacts to fabric tape, the contacts electrically coupled to the resistive material. Any method can be used to affix the contacts to the fabric tape and electrically couple the contacts to the resistive material. In some embodiments, two different mechanisms may be used, one mechanism to mechanically affix the contacts to the fabric tape and a different mechanism to electrically couple the contacts to the resistive material. In other embodiments, a single mechanism may accomplish both affixing the contacts to the fabric tape and electrically coupling the contact to the resistive material. Various embodiments may use any appropriate mechanism including, but not limited to, sewing, riveting, gluing, weaving, embedding contacts in wet conductive ink and allowing the conductive ink to cure, spring-loaded pincers, pins, or screws.

In some embodiments the electrical contacts are spaced at a fixed interval longitudinally on the fabric tape. In at least one embodiment, the electrical contacts use conductive loop fasteners or conductive hook fasteners. The heating kinesiology tape has been successfully manufactured at block 709.

It should also be noted that, in some alternative implementations, not all the activities shown are performed and/or the activities noted in the block may occur out of the order noted in the flowchart 600 or the flowchart 700. For example, activities described in two separate blocks may, in fact, be performed substantially concurrently, or the activities described in two separate blocks may be executed in reverse order. It will also be noted that each block may be performed by one entity, or by different entities in any combination.

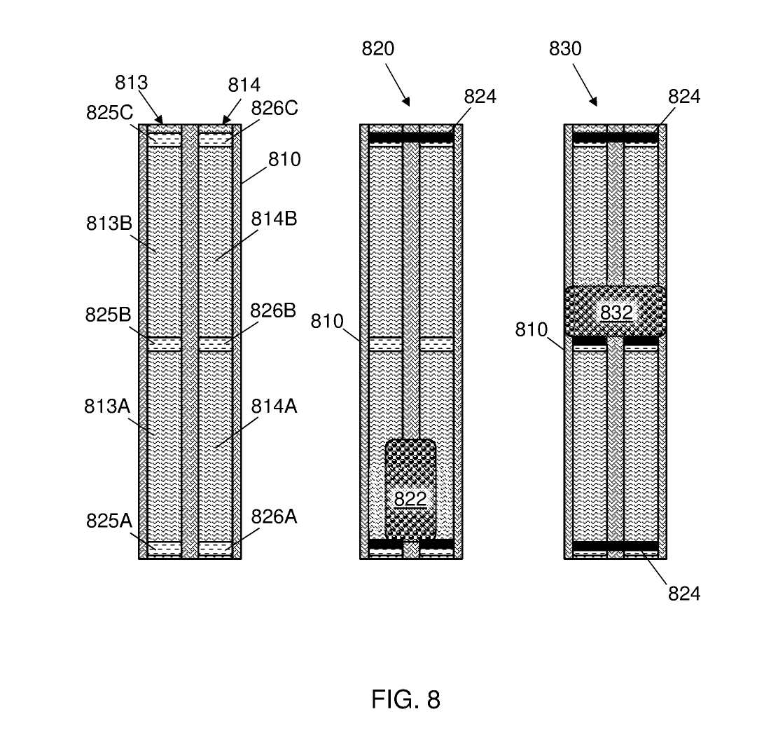

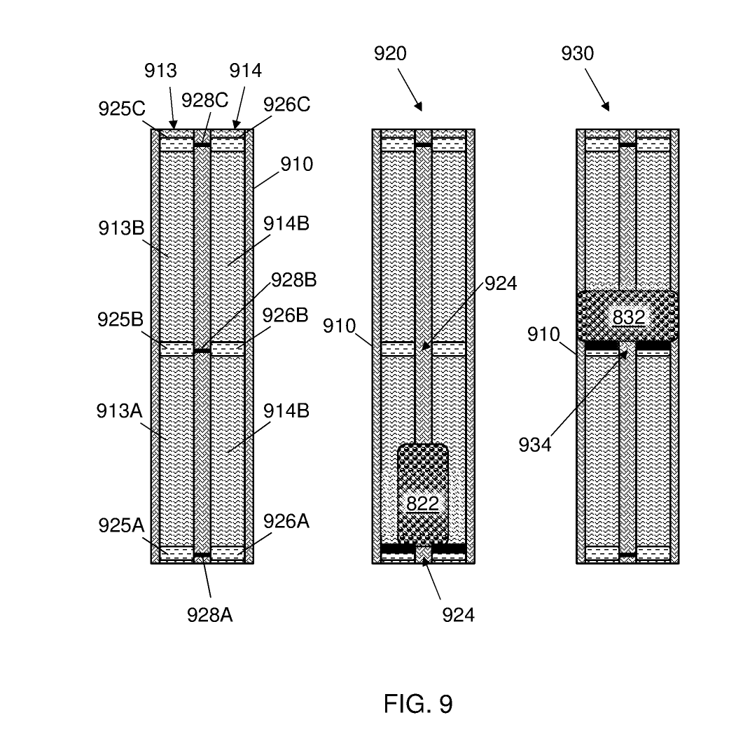

In some embodiments, such as those shown in FIG. 8 and FIG. 9, the heating kinesiology tape 810/910 may include two parallel heating elements, 813/913 and 814/914, extending the length of the tape with pairs of electrical contacts, such as contact 825A/925A electrically coupled to heating element 813/913 and contact 826A/926A electrically coupled to heating element 814/914, spaced at a regular intervals along the tape, where each interval can be thought of as a segment of tape. In such embodiments, a power supply 822/832 may be provided that has two source contacts adapted to be electrically coupled to a pair of electrical contacts, such as contacts 825/925A and 826A/926A, on the heating kinesiology tape 810/910. A particular power supply may be designed to power a particular number of segments of tape.

In some embodiments, such as the embodiment shown in FIG. 8, the two electrical contacts in a pair of electrical contacts, such as contacts 825C and 826C, are not electrically connected on the heating kinesiology tape 810, and one or more shunts 824 are provided that each have two electrically connected contacts adapted to be electrically coupled to a pair of electrical contacts, such as contacts 825C and 826C, on the heating kinesiology tape 810 as shown in heating kinesiology tape system 820. The power supply 822/832 can then be coupled to one pair of electrical contacts on the heating kinesiology tape 810 and the one or more shunts 824 can then be coupled to one or more other pairs of electrical contacts on the heating kinesiology tape 810 to complete the circuit so that the power supply 822/832 can provide electrical power to the heating elements 813, 814 to generate heat.

In other embodiments, such as the embodiment shown in FIG. 9, the heating kinesiology tape 910 may include shunts between some of the pairs of electrical contacts, such as shunt 928A coupling electrical contact 925A to electrical contact 926A, that may be removed, separately cut, cut by cutting the tape between the contacts, or otherwise disabled. At the time of application of the heating kinesiology tape 910, one or more of the shunts 928A-C can be disabled and the power supply 822/832 can be coupled to a pair of electrical contacts with a disabled shunt. One or more remaining shunts can complete the circuit so that the power supply 822/832 can provide electrical power to the heating elements to generate heat.

As an example, a power supply 822 with a voltage suitable to power two serially coupled sections of tape is obtained along with a piece of heating kinesiology tape 810/910 having a pair of electrical contacts 825A/925A and 826B/926B near a first end, another pair of electrical contacts 825A/925A and 826B/926B near the middle, and a pair of electrical contacts 825C/925C and 826C/926C near a second end, providing two segments of the tape 810/910. If shunts are not incorporated into the heating kinesiology tape 810, as shown in FIG. 8, a shunt 824 may be coupled to a pair of electrical contacts 825C, 826C near one end of the heating kinesiology tape 810, and the power supply 822 may be coupled to the pair of electrical contacts 8c5A, 826A near the other end of the heating kinesiology tape 810, for heating kinesiology tape system 820. If the shunts are incorporated into the heating kinesiology tape 910, as shown in FIG. 9, a shunt 928C may be left coupling a pair of electrical contacts 925C, 926C near one end of the heating kinesiology tape and the other two shunts 928A, 928B disabled 924 by removing or cutting the shunts 928A, 928B. The power supply 822 is then coupled to the pair of electrical contacts 925A, 926A near the other end of the heating kinesiology tape 910, for heating kinesiology tape system 920.

In another example, a power supply 832 with a voltage suitable to power two parallel coupled sections of tape is obtained along with a piece of heating kinesiology tape 810/910 having a pair of electrical contacts 825A/925A and 826B/926B near a first end, another pair of electrical contacts 825A/925A and 826B/926B near the middle, and a pair of electrical contacts 825C/925C and 826C/926C near a second end, providing two segments of the tape 810/910. If shunts are not incorporated into the heating kinesiology tape 810, as shown in FIG. 8, shunts 824 may be coupled to the two pairs of electrical contacts 825A, 826A and 825C, 826C near the ends of the heating kinesiology tape 810, and the power supply 832 may be coupled to the pair of electrical contacts 825B, 826B near the middle of the heating kinesiology tape 810, for heating kinesiology tape system 830. If the shunts are incorporated into the heating kinesiology tape 910, the shunt 928B coupling the middle pair of electrical contacts 925B, 926B can be disabled 934 by removing or cutting the shunt 928B, and the power supply 832 may be coupled to the middle pair of electrical contacts 925B, 926B of the heating kinesiology tape 910, for heating kinesiology tape system 930.

Other embodiments using heating kinesiology tape 810/910 with two parallel heating elements extending the length of the tape may include power supplies designed to power other configurations of segments of tape. If one segment has a resistance of 1 ohm (Q), and one watt (W) per segment is desired, a voltage regulated power supply to power one segment would need to provide 1 volt (V) which would lead to a 1 ampere (A) current. For two segments in series as shown in systems 820/920, the power supply 924 would need to provide 2V to the 2Ω presented by the two serially connected segments of tape, leading to 1 W of power to each section. For two segments in parallel as shown in systems 830/930, the power supply 832 would need to provide 1V to the 0.5Ω presented by the two parallel segments of tape, leading to 2 A total (1 A for each section), to get 1 W of power to each section). If an equal amount of heat per unit area is required throughout the length of the tape, other topologies are possible, including any number of serially connected segments, or two parallel connected multi-segment loads with equal numbers of segments. If, however, the number of segments on the two sides of the power supply are not equal, creating two parallel connected heating elements with different resistances, the heating of the two portions of the tape will be unequal, with the shorter heating element (with lower resistance) providing more heat per unit area than the longer heating element (with higher resistance).

In other embodiments, the two heating elements 813/913, 814/914, don't have equivalent resistance, and the heating may differ between the first heating element 813/913 and the second heating element 814/914. In at least one embodiment, the first heating element 813/913 has a very low resistance and can act as an electrical conductor from an electrical contact of the power supply 822/832 to one or more contacts of the second heating element 814/914. In such an embodiment, the heat is generated by the second heating element 814/914 with little to no heat generated by the very low resistance first heating element 813/913.

Examples of various embodiments are described in the following paragraphs:

An example adhesive tape includes an anisotropically stretchable fabric material having a first side and a second side opposite the first side, a pressure sensitive adhesive fixed on the first side of the fabric material, a heating element coupled to the fabric material, and electrical contacts electrically coupled to the heating element and accessible from the second side of the fabric material. In some example adhesive tapes, the electrical contacts include a first electrical contact and a second electrical contact, wherein heat is generated by the heating element when an electrical voltage is applied between the first electrical contact and the second electrical contact. In some example adhesive tapes, the anisotropically stretchable fabric material is stretchable longitudinally, and the heating element includes a serpentine shaped resistive element extending longitudinally along the adhesive tape. In some example adhesive tapes, the serpentine shaped resistive element retains electrical continuity when the adhesive tape is stretched to 180% of its original length. In some example adhesive tapes, the electrical contacts include three or more electrical contacts distributed substantially equally along the adhesive tape longitudinally. In some example adhesive tapes, the electrical contacts include three or more electrical contacts spaced along the unstretched adhesive tape substantially at a predetermined distance apart. In some example adhesive tapes, the electrical contacts are provided separately from the fabric material and adapted to be electrically coupled to the heating element by an individual at a time of application of the adhesive tape to a human body. In some example adhesive tapes, the heating element includes conductive fibers woven into the fabric material. In some example adhesive tapes, the heating element includes conductive fibers affixed to the fabric material. In some example adhesive tapes, the heating element includes conductive ink applied to the fabric material. Some adhesive tapes also include an insulating layer affixed to the second side of the fabric material and covering the heating element, the insulating layer having openings to expose the electrical contacts. In some example adhesive tapes, the pressure sensitive adhesive has electrical insulating properties. In some example adhesive tapes, the heating element is positioned between the pressure sensitive adhesive and the fabric material. In some example adhesive tapes, the pressure sensitive adhesive includes the heating element. In some example adhesive tapes, the electrical contacts include conductive loop fasteners or conductive hook fasteners. In some example adhesive tapes, the electrical contacts include conductive snap fasteners. In some example adhesive tapes, the electrical contacts include conductive surfaces adapted for use with conductive glue. Any combination of the examples of this paragraph may be used in embodiments.

An example heating tape system includes an adhesive tape and a power supply. In the example heating tape system, the adhesive tape includes an anisotropically stretchable fabric material having a first side and a second side opposite the first side, a pressure sensitive adhesive fixed on the first side of the fabric material, a heating element coupled to the fabric material, and a first load contact and a second load contact, both electrically coupled to the heating element and accessible from the second side of the fabric material. In the example heating tape system, the power supply includes a source of electrical energy comprising a first terminal and a second terminal, a first source contact electrically coupled to the first terminal of the source of electrical energy and adapted to be electrically coupled to the first load contact, and a second source contact electrically coupled to the second terminal of the source of electrical energy and adapted to be electrically coupled to the second load contact. In some example heating tape systems, the source of electrical energy includes a battery or a battery holder. In some example heating tape systems, the source of electrical energy includes a fixed current power source. In some example heating tape systems, the power supply further includes a fuse coupled between the source of electrical energy and the first source contact. In some example heating tape systems, the power supply further includes an on/off switch coupled between the source of electrical energy and the first source contact. In some example heating tape systems, the power supply further includes a wire electrically coupling the source of electrical energy to the first source contact. In some example heating tape systems, the source of electrical energy includes a fixed voltage source having a voltage based on a targeted length of the adhesive tape, and a length of the wire electrically coupling the source of electrical energy to the first source contact is based on the targeted length of the adhesive tape. Some example heating tape systems also include conductive glue to electrically couple the first source contact to the first load contact and the second source contact to the second load contact. In some example heating tape systems, the adhesive tape further includes a power supply attachment area with loop fasteners located at a first distance from the first load contact on the unstretched adhesive tape, and conductive loop fasteners on the first load contact. In some example heating tape systems, the power supply further includes a case having hook fasteners to mate with the loop fasteners of the power supply attachment space on the adhesive tape, conductive hook fasteners on the first source contact to mate with the conductive loop fasteners of the first load contact, and a conductor extending from the case electrically coupling the source of electrical energy to the first source contact, the conductor having a length based on the first distance and a stretch factor for the adhesive tape. In some example heating tape systems, the power supply attachment area is located between the first load contact and the second load contact. In some example heating tape systems, the adhesive tape further includes conductive loop fasteners in the power supply attachment area to act as the second load contact, and the power supply further includes conductive hook fasteners on the case to act as the second source contact to mate with the conductive loop fasteners of the second load contact. In some example heating tape systems, the power supply is a first power supply and the system also includes a second power supply. In some example heating tape systems, the adhesive tape also includes a third load contact coupled to the heating element and accessible from the second side of the fabric material, the third load contact comprising conducting loop fasteners and located at the first distance from the second load contact opposite from the first load contact. In some example heating tape systems, the second power supply includes a second case having conductive hook fasteners to act as a third source contact and to mate with the conductive loop fasteners of the third load contact on the adhesive tape, a second source of electrical energy coupled to the third source contact, a fourth source contact comprising conductive hook fasteners to mate with the conductive loop fasteners of the first load contact, and a second conductor extending from the second case electrically coupling the second source of electrical energy to the fourth source contact, the second conductor having a length of about twice the length of the conductor of the first power supply. In some example heating tape systems, the source of electrical energy of the first power supply has a first voltage, and the second source of electrical energy has a second voltage about twice that of the first voltage. In some example heating tape systems, the source of electrical energy of the first power supply utilizes a single battery, and the second source of electrical energy utilizes two batteries in series. In some example heating tape systems, the power supply further includes additional conductive loop fasteners, on a different side of the case from the second source contact, to act as a daisy-chain contact, the daisy-chain contact electrically coupled to the second source contact. In some example heating tape systems, the second power supply is substantially the same as the first power supply. In some example heating tape systems, the adhesive tape also includes a third load contact coupled to the heating element and accessible from the second side of the fabric material, the third load contact comprising conducting loop fasteners and located at the first distance from the second load contact opposite from the first load contact. In some example heating tape systems, the conductive hook fasteners on the case of the second power supply act as the second source contact of the second power supply and are adapted to mate with the conductive loop fasteners of the third load contact, and the conductive hook fasteners on the first source contact of the second power supply are adapted to mate with the conductive loop fasteners of the daisy-chain contact of the first power supply. Any combination of the examples of this paragraph may be used in embodiments.

An example method of applying heating kinesiology tape to a human body includes identifying a portion of a human body for treatment with heating kinesiology tape, obtaining piece of heating kinesiology tape of an appropriate length for treating the identified portion of the human body, the piece of heating kinesiology tape comprising an anisotropically stretchable fabric material, a pressure sensitive adhesive fixed to the fabric material, a heating element coupled to the fabric material, and a first load contact and a second load contact, both electrically coupled to the heating element, stretching the piece of heating kinesiology tape to at least 125% of its unstretched length, applying the stretched piece of heating kinesiology tape to the identified portion of the human body, and attaching a first source contact of a power supply to the first load contact, and a second source contact of the power supply to the second load contact to create an electrical circuit comprising the power supply and the heating element. Some example methods of applying also include turning on the power supply to provide electrical current from the power supply to the heating element. In some example methods of applying, the stretching includes stretching the piece of heating kinesiology tape to between 130% and 175% of its unstretched length. Some example methods of applying also include selecting the power supply from a plurality of power supplies based on a stretched length of the piece of heating kinesiology tape. In some example methods of applying, the selecting the power supply from a plurality of power supplies is also based on a length of a conductor coupling the first source contact to the selected power supply. Some example methods of applying also include coupling the power supply to the human body. In some example methods of applying, the applying the heating kinesiology tape includes positioning one end of the piece of heating kinesiology tape at the origin of a muscle in the identified portion of the human body, and positioning an opposite end of the piece of heating kinesiology tape at the insertion point of the muscle. Any combination of the examples of this paragraph may be used in embodiments.

An example method of manufacturing heating kinesiology tape includes (a) obtaining anisotropically stretchable fabric tape, the fabric tape longitudinally stretchable, (b) stretching a portion of the fabric tape longitudinally, (c) applying resistive material to the stretched portion of the fabric tape, (d) unstretching the stretched portion of the fabric tape, and (e) applying pressure sensitive adhesive to the fabric tape. Some example methods of manufacturing also include keeping the stretched portion of the fabric stretched for a curing time of the resistive material. Some example methods of manufacturing also include applying an insulating layer on top of the resistive material with a gap in the insulating layer at a regular interval to expose the resistive material. Some example methods of manufacturing also include repeating (b) through (e) with successive portions of the fabric tape in a continuous process. Some example methods of manufacturing also include affixing contacts to fabric tape, the contacts electrically coupled to the resistive material. In some example methods of manufacturing, the electrical contacts are spaced at a fixed interval longitudinally on the fabric tape. In some example methods of manufacturing, the electrical contacts include conductive loop fasteners or conductive hook fasteners. Any combination of the examples of this paragraph may be used in embodiments.

The description of the various embodiments provided above is illustrative in nature and is not intended to limit the invention, its application, or uses. Thus, different variations beyond those described herein are intended to be within the scope of the embodiments of the present invention. Such variations are not to be regarded as a departure from the intended scope of the present invention. As such, the breadth and scope of the present invention should not be limited by the above-described exemplary embodiments, but should be defined only in accordance with the following claims and equivalents thereof.

Unless otherwise indicated, all numbers expressing quantities of elements, optical characteristic properties, and so forth used in the specification and claims are to be understood as being modified in all instances by the term “about.” Accordingly, unless indicated to the contrary, the numerical parameters set forth in the preceding specification and attached claims are approximations that can vary depending upon the desired properties sought to be obtained by those skilled in the art utilizing various principles of the present disclosure. Recitation of numerical ranges by endpoints includes all numbers subsumed within that range (e.g. 1 to 5 includes 1, 2.78, π, and 5). As used in this specification and the appended claims, the singular forms “a”, “an”, and “the” include plural referents unless the content clearly dictates otherwise. Thus, for example, reference to an element described as “an opening” may refer to a single opening, two openings, or any other number of openings. As used in this specification and the appended claims, the term “or” is generally employed in its “and/or” inclusive sense, which includes the case where all the elements are included, unless the content clearly dictates otherwise. As used herein, the term “coupled” includes direct and indirect connections. Moreover, where first and second devices are coupled, intervening elements including active elements may be located there between. Any element in a claim that does not explicitly state “means for” performing a specified function, or “step for” performing a specified function, is not to be interpreted as a “means” or “step” clause as specified in 35 U.S.C. § 112(f).

The description of the various embodiments provided above is illustrative in nature and is not intended to limit the present invention, its application, or uses. As such, the breadth and scope of the present invention should not be limited by the above-described embodiments, but should be defined only in accordance with the following claims and equivalents thereof.

1. An adhesive tape comprising:
an anisotropically stretchable fabric material having a first side and a second side opposite the first side;
a pressure sensitive adhesive fixed on the first side of the fabric material;
a heating element coupled to the fabric material; and
electrical contacts electrically coupled to the heating element and accessible from the second side of the fabric material, the electrical contacts comprising conductive snap fasteners, conductive loop fasteners or conductive hook fasteners.

2. The adhesive tape of claim 1, the electrical contacts comprising a first electrical contact and a second electrical contact, wherein heat is generated by the heating element when an electrical voltage is applied between the first electrical contact and the second electrical contact.

3. The adhesive tape of claim 1, the anisotropically stretchable fabric material stretchable longitudinally, the heating element comprising a serpentine shaped resistive element extending longitudinally along the adhesive tape.

4. The adhesive tape of claim 3, wherein the serpentine shaped resistive element retains electrical continuity when the adhesive tape is stretched to 180% of its original length.

5. (canceled)

6. The adhesive tape of claim 1, the electrical contacts comprising three or more electrical contacts spaced along the unstretched adhesive tape substantially at a predetermined distance apart.

7. The adhesive tape of claim 1, the electrical contacts provided separately from the fabric material and adapted to be electrically coupled to the heating element by an individual at a time of application of the adhesive tape to a human body.

8-9. (canceled)

10. The adhesive tape of claim 1, the heating element comprising conductive ink applied to the fabric material.

11. The adhesive tape of claim 1, further comprising:
an insulating layer affixed to the second side of the fabric material and covering the heating element, the insulating layer having openings to expose the electrical contacts.

12. The adhesive tape of claim 1, the pressure sensitive adhesive having electrical insulating properties.

13. The adhesive tape of claim 12, the heating element positioned between the pressure sensitive adhesive and the fabric material.

14. The adhesive tape of claim 1, the pressure sensitive adhesive comprising the heating element.

15-17. (canceled)

18. A heating tape system comprising an adhesive tape and a power supply;
the adhesive tape comprising:
an anisotropically stretchable fabric material having a first side and a second side opposite the first side;
a pressure sensitive adhesive fixed on the first side of the fabric material;
a heating element coupled to the fabric material; and
a first load contact and a second load contact, both electrically coupled to the heating element and accessible from the second side of the fabric material, the first load contact and the second load contact each comprising conductive snap fasteners, conductive loop fasteners or conductive hook fasteners;
the power supply comprising:
a source of electrical energy comprising a first terminal and a second terminal;
a first source contact comprising conductive snap fasteners, conductive loop fasteners or conductive hook fasteners, the first source contact electrically coupled to the first terminal of the source of electrical energy and adapted to be electrically coupled to the first load contact; and
a second source contact comprising conductive snap fasteners, conductive loop fasteners or conductive hook fasteners, the second source contact electrically coupled to the second terminal of the source of electrical energy and adapted to be electrically coupled to the second load contact.

19. The system of claim 18, the source of electrical energy comprising a battery or a battery holder.

20. The system of claim 18, the source of electrical energy comprising a fixed current power source.

21-23. (canceled)

24. The system of claim 18, the power supply further comprising a wire electrically coupling the source of electrical energy to the first source contact;
the source of electrical energy comprising a fixed voltage source having a voltage based on a targeted length of the adhesive tape, and a length of the wire electrically coupling the source of electrical energy to the first source contact is based on the targeted length of the adhesive tape.

25. (canceled)

26. The system of claim 18, the adhesive tape further comprising:
a power supply attachment area with loop fasteners located at a first distance from the first load contact on the unstretched adhesive tape; and
the first load contact comprising conductive loop fasteners;
the power supply further comprising:
a case having hook fasteners to mate with the loop fasteners of the power supply attachment area on the adhesive tape; and
conductive hook fasteners on the first source contact to mate with the conductive loop fasteners of the first load contact; and
a conductor extending from the case electrically coupling the source of electrical energy to the first source contact, the conductor having a length based on the first distance and a stretch factor for the adhesive tape;
the first source contact comprising conductive hook fasteners to mate with the conductive loop fasteners of the first load contact.

27. The system of claim 26, the power supply attachment area located between the first load contact and the second load contact.

28. The system of claim 26, the adhesive tape further comprising conductive loop fasteners in the power supply attachment area to act as the second load contact; and
the power supply further comprising conductive hook fasteners on the case to act as the second source contact to mate with the conductive loop fasteners of the second load contact.

29. The system of claim 28, wherein the power supply is a first power supply;
the system further comprising a second power supply;
the adhesive tape further comprising:
a third load contact coupled to the heating element and accessible from the second side of the fabric material, the third load contact comprising conducting loop fasteners and located at the first distance from the second load contact opposite from the first load contact;
the second power supply comprising:
a second case having conductive hook fasteners to act as a third source contact and to mate with the conductive loop fasteners of the third load contact on the adhesive tape;
a second source of electrical energy coupled to the third source contact;
a fourth source contact comprising conductive hook fasteners to mate with the conductive loop fasteners of the first load contact; and
a second conductor extending from the second case electrically coupling the second source of electrical energy to the fourth source contact, the second conductor having a length of about twice the length of the conductor of the first power supply.

30. The system of claim 29, wherein the source of electrical energy of the first power supply has a first voltage, and the second source of electrical energy has a second voltage about twice that of the first voltage.

31. The system of claim 29, wherein the source of electrical energy of the first power supply utilizes a single battery, and the second source of electrical energy utilizes two batteries in series.

32. The system of claim 28, the power supply further comprising additional conductive loop fasteners, on a different side of the case from the second source contact, to act as a daisy-chain contact, the daisy-chain contact electrically coupled to the second source contact.

33. The system of claim 32, wherein the power supply is a first power supply;
the system further comprising a second power supply substantially the same as the first power supply;
the adhesive tape further comprising:
a third load contact coupled to the heating element and accessible from the second side of the fabric material, the third load contact comprising conducting loop fasteners and located at the first distance from the second load contact opposite from the first load contact;
wherein the conductive hook fasteners on the case of the second power supply act as the second source contact of the second power supply and are adapted to mate with the conductive loop fasteners of the third load contact, and the conductive hook fasteners on the first source contact of the second power supply are adapted to mate with the conductive loop fasteners of the daisy-chain contact of the first power supply.

34. A method of applying heating kinesiology tape to a human body, the method comprising:
identifying a portion of a human body for treatment with heating kinesiology tape;
obtaining a piece of heating kinesiology tape of an appropriate length for treating the identified portion of the human body, the piece of heating kinesiology tape comprising an anisotropically stretchable fabric material, a pressure sensitive adhesive fixed to the fabric material, a heating element coupled to the fabric material, and a first load contact and a second load contact, both electrically coupled to the heating element, the first load contact and the second load contact each comprising conductive snap fasteners, conductive loop fasteners or conductive hook fasteners;
stretching the piece of heating kinesiology tape to at least 125% of its unstretched length;
applying the stretched piece of heating kinesiology tape to the identified portion of the human body; and
attaching a first source contact, comprising a fastener adapted to mate with the first load contact, of a power supply to the first load contact, and a second source contact, comprising a fastener adapted to mate with the second load contact, of the power supply to the second load contact to create an electrical circuit comprising the power supply and the heating element.

35-36. (canceled)

37. The method of claim 34, further comprising selecting the power supply from a plurality of power supplies based on a stretched length of the piece of heating kinesiology tape.

38. The method of claim 37, wherein the selecting the power supply from a plurality of power supplies is also based on a length of a conductor coupling the first source contact to the selected power supply.

39. The method of claim 34, further comprising coupling the power supply to the human body.

40. The method of claim 34, the applying the stretched piece of heating kinesiology point comprising:
positioning one end of the piece of heating kinesiology tape at the origin of a muscle in the identified portion of the human body; and
positioning an opposite end of the piece of heating kinesiology tape at the insertion point of the muscle.

41-88. (canceled)
